SEQUENCE LISTING (22 sequences)

SEQ ID NO: 1 - DNA, 20 bp, Escherichia coli
tctcagtgggcgttcttatg

SEQ ID NO: 2 - DNA, 20 bp, Escherichia coli
taccccctcaactgctaata

SEQ ID NO: 3 - DNA, 21 bp, Escherichia coli
tgtcttcagcatcttatgcag

SEQ ID NO: 4 - DNA, 25 bp, Escherichia coli
catgattaattactgaaacagaaac

SEQ ID NO: 5 - DNA, 20 bp, Escherichia coli
gcggttttatttgcattagc

SEQ ID NO: 6 - DNA, 20 bp, Escherichia coli
tcccgtcaaccttcactgta

SEQ ID NO: 7 - DNA, 20 bp, Escherichia coli
gcggttttatttgcattagt

SEQ ID NO: 8 - DNA, 20 bp, Escherichia coli
agtactcttttccggccact

SEQ ID NO: 9 - DNA, 25 bp, Escherichia coli
atgaagtgtatattgttaaagtgga

SEQ ID NO: 10 - DNA, 22 bp, Escherichia coli
agccacatataaattatttcgt

SEQ ID NO: 11 - DNA, 20 bp, Escherichia coli
atgcttagtgctggtttagg

SEQ ID NO: 12 - DNA, 20 bp, Escherichia coli
gccttcatcatttcgctttc

SEQ ID NO: 13 - DNA, 24 bp, Escherichia coli
ggtaaaattgagttctctaagtat

SEQ ID NO: 14 - DNA, 21 bp, Escherichia coli
cagcaaatcctgaacctgacg

SEQ ID NO: 15 - DNA, 19 bp, Escherichia coli
agctgcaagtgcgggtctg

SEQ ID NO: 16 - DNA, 24 bp, Escherichia coli
tacgggttatgcctgcaagttcac

SEQ ID NO: 17 - DNA, 21 bp, Escherichia coli
ctacaggtgaaggtggaatgg

SEQ ID NO: 18 - DNA, 21 bp, Escherichia coli
attcctctctttcctctgcgg

SEQ ID NO: 19 - DNA, 21 bp, Escherichia coli
taccatcgcaaaagcaactcc

SEQ ID NO: 20 - DNA, 21 bp, Escherichia coli
gtcggcaacgttagtgatacc

SEQ ID NO: 21 - DNA, 20 bp, Escherichia coli
ccccctggacgaagactgac

SEQ ID NO: 22 - DNA, 20 bp, Escherichia coli
accgctggcaacaaaggata

(19) United States
(12) Patent Application Publication
(10) Pub. No.: US 2006/0051751 A1
(43) Pub. Date: Mar. 9, 2006

(54) Major virulence factor detection and verocytontoxin type 2 subtype from clinical e. coli isolates using a one-step multiplex pcr

(76) Inventors: Gehua Wang, Manitoba (CA); Frank G Rodgers, Manitoba (CA)

Correspondence Address: Ade & Company, 1700-360 Main Street, Winnipeg, R3C 3Z3, CA

(21) Appl. No.: 10/502,314

(22) PCT Filed: Jan. 7, 2003
(86) PCT No.: PCT/CA03/00042

§ 371 Date: Apr. 29, 2005

Related U.S. Application Data
(60) Provisional application No. 60/349,981, filed on Jan. 23, 2002.

(51) Int. Cl. C12Q 1/68; C07H 21/04

(52) U.S. Cl. 435/6; 536/24.1

(57) ABSTRACT

A single kit comprising 3 multiplex PCR assays that can detect in E. coli the presence of the 8 virulence genes: eaeA, EHEC-HlyA, Stx1 (VT1), Stx2 (VT2), Stx2c (VT2c), Stx2d (VT2d), Stx2e (VT2e) and Stx2f (VT2f) is described. In addition, the kit can detect the two critical serotypes (O157 and H7) and identify the species (Escherichia coli) simultaneously using a one step reaction. Following evaluation in our hands, this PCR kit has been used to detect the above 11 components of disease-causing E. coli in a fast, accurate, reliable and specific fashion. These kits can be used on bacterial isolates and has the potential for use directly on foods and environmental samples.

### FIELD OF THE INVENTION

The present invention relates generally to the field of pathogenic organisms. More specifically, the present invention relates to a multiplex PCR-based method for identifying and characterizing E. coli strains.

### BACKGROUND OF THE INVENTION

Rapid identification of Escherichia coli O157:H7 infection is important because medicines that may be given for similar syndromes can trigger kidney complications and lead to hemolytic uremic syndrome (HUS). The expression products of the VT-2 toxin family along with the eae and hemolysin genes are closely associated with disease induction. Therefore, their detection is crucial to impacting morbidity caused by this pathogen and to reducing the economic burden brought about by this disease.

Currently, there is no specific treatment for HUS. Therefore, there is an urgent need for preventative measures that are based on a detailed understanding of the epidemiology of verotoxin-producing E. coli (VTEC) infections. Such measures will also be dependent on the rapid availability of rapid, sensitive, simple, reproducible and cost effective procedures for the detection of these pathogens and their toxins from humans and animals as well as from samples such as food and water.

E. coli O157:H7 (Verotoxin-producing E. coli) was first recognized in 1982 following an outbreak of hemorrhagic colitis (HC) in the US. However, it was the notorious outbreak associated with a fast-food restaurant chain in the US in 1993 that catapulted the pathogen into the public limelight. The Centre for Disease Control now estimates that in the US this strain causes 73,000 illnesses and in excess of 60 deaths each year. Verotoxins produced by VTEC strains may result in life-threatening complications such as HUS. In addition, several putative accessory virulence factors have been identified and partly characterized. These include:

Attaching and effacing adherence (eaeA)—A strong link has been confirmed between the expression of the eaeA gene product and the capacity of VTEC strains to cause severe human disease such as HC and HUS (Paton and Paton, 1998, Clin Microb Rev 11: 450-479).

Enterohemorrhagic E. coli (EHEC) hemolysin (EHEC-HlyA)-EHEC-HlyA is the structural gene for the hemolysin. Investigations (Schmidt et al., 1995, Infect Immun 63: 1055-1061) indicated that the EHEC-HlyA has clinical importance. This hemolysin occurs in all O157 strains tested and is reactive to sera of patients with HUS (i.e. patients with HUS develop antibodies to the hemolysin).

Verocytotoxins—VTs were classified in two major classes: VT1 and VT2 (Paton and Paton, 1998; Schmidt et al., 1995; Pierard et al., 1998, J Clin Microbiol 36: 3317-3322). Although the VT1 class is highly homogeneous, five subtypes of VT2 have been identified: VT2, VT2c, VT2d, VT2e and VT2f. Within the VTEC family, certain strains appear to have greater virulence for humans. Comparative studies suggested that naturally occurring VT2 sequence variations may impact directly on the capacity of a given VT-producing E. coli strain to cause disease (Paton et al., 1995, Infect Immun 63: 2450-2458). Current data suggest that toxin type secreted by strains of E. coli is an important factor in the probability of developing HUS or other severe complications associated with this disease. However, nucleotide and deduced amino acid sequence analysis of the VT2 family of toxins showed that they are highly conserved (82.8 to 99.3% similarity) (Ito et al., 1990, Microb Pathog 8: 47-60) make it difficult to differentiate between VT2 variants using PCR alone. To date, there are several multiplex PCR assays already developed to detect virulence factors in EHEC. Most target the VT genes (VT1 and VT2) either alone or in combination with eaeA, EHEC-hlyA or the O157:H7 serotype (Pass et al., 2000, J Clin Microbiol 38: 2001-2004; Feng and Monday, 2000, Mol Cell Probes 14: 333-337; Hu et al., 1999, J Appl Microbiol 87: 867-876). For the differentiation of human VT2 variants, the most widely used genotyping method is based on restriction fragment length polymorphism (RFLP) analysis; however, this process is expensive and time-consuming (Peirard et al., 1998; Tyler et al., 1991, J Clin Microbiol 29: 1339-1343).

Applied Biosystems (formerly Perkin Elmer), Dupont and Panvera have released O157:H7 and/or VT toxin detection kits. It was anticipated that these kits would be used by public health laboratories to help control outbreaks and perform epidemiologic studies. In addition, it was felt that the food industry and government laboratories would also use these products to define food testing practices and facilitate food recall activities. Finally, academic researchers would use these kits to investigate virulence factors associated with the pathogen. However, none of these kits can be used to subtype the VT2 toxin family.

U.S. Pat. No. 5,747,257 describes the use of random amplified polymorphic DNA amplification to discover fragments diagnostic of E. coli O157:H7 serotypes. Probes derived from these sequences can then be used to identify E. coli O157:H7 strains. However, this method does not provide any information on verotoxin subtype or presence of eaeA.

U.S. Pat. No. 5,654,417 also teaches the use of a region of the E. coli O157:H7 genome for generating probes for screening for O157:H7 serotype. However, this method does not provide any information on verotoxin subtype or presence of eaeA.

U.S. Pat. No. 5,756,293 provides the sequence of the HlyA and HlyB genes as well as the region therebetween and describes the use of primers and probes derived from these sequences for the detection of enterohemorrhagic E. coli. However, specific primers for use in a multiplex system for further characterizing the E. coli strain is not taught or disclosed.

U.S. Pat. No. 6,291,168 describes the isolation of a unique DNA sequence found in E. coli O157:H7 isolates and the use of same for the identification of O157:H7 isolates. However, this method does not provide any information on verotoxin subtype or presence of eaeA.

U.S. Pat. No. 6,268,143 describes a PCR-based 5′nuclease assay using the eaeA sequence for detecting E. coli O157:H7. However, specific primers for use in a multiplex system for further characterizing the E. coli strain is not taught or disclosed.

U.S. Pat. No. 6,162,605 teaches the use of strand displacement amplification in combination with assay probes derived from SLT-I conserved DNA regions to identify samples containing SLT-I. However, use of the conserved regions of SLT-I means that subtyping of verotoxins is not possible.

U.S. Pat. No. 5,652,102 teaches the use of primers derived from a 60 Mda plasmid present in most EHEC strains for identifying E. coli O157:H7. These primers are used in a multiplex kit along with primers derived from SLT conserved regions and eaeA. As discussed above, this method does not provide information on HlyA or verotoxin subtypes.

Clearly, a multiplex PCR system for detecting the presence of E. coli virulence genes eaeA, EHEC-HlyA, Stx1 (VT1), Stx2 (VT2), Stx2c (VT2c), Stx2d (VT2d), Stx2e (VT2e) and Stx2f (VT2f) and the serotypes O157 and H7 simultaneously using a one step reaction is needed.

### SUMMARY OF THE INVENTION

According to a first aspect of the invention, there is provided a pair of primers selected from the group consisting of at least 15 contiguous nucleotides of: TCTCAGTGGGCGTTCTTATG (SEQ ID NO. 1) and TACCCCCTCAACTGCTAATA (SEQ ID NO. 2); TGTCTTCAGCATCTTATGCAG (SEQ ID NO. 3) and CATGATTAATTACTGAAACAGAAAC (SEQ ID NO. 4); GCGGTTTTATTTGCATTAGC (SEQ ID NO. 5) and TCCCGTCAACCTTCACTGTA (SEQ ID NO. 6); GCGGTTTTATTTGCATTAGT (SEQ ID NO. 7) and AGTACTCTTTTCCGGCCACT (SEQ ID NO. 8); ATGAAGTGTATATTGTTAAAGTGGA (SEQ ID NO. 9) and AGCCACATATAAATTATTTCGT (SEQ ID NO. 10); ATGCTTAGTGCTGGTTTAGG (SEQ ID NO. 11) and GCCTTCATCATTTCGCTTTC (SEQ ID NO. 12); GGTAAAATTGAGTTCTCTAAGTAT (SEQ ID NO. 13) and CAGCAAATCCTGAACCTGACG (SEQ ID NO. 14); AGCTGCAAGTGCGGGTCTG (SEQ ID NO. 15) and TACGGGTTATGCCTGCAAGTTCAC (SEQ ID NO. 16); CTACAGGTGAAGGTGGAATGG (SEQ ID NO. 17) and ATTCCTCTCTTTCCTCTGCGG (SEQ ID NO. 18); TACCATCGCAAAAGCAACTCC (SEQ ID NO. 19) and GTCGGCAACGTTAGTGATACC (SEQ ID NO. 20); CCCCCTGGACGAAGACTGAC (SEQ ID NO. 21) and ACCGCTGGCAACAAAGGATA (SEQ ID NO. 22) and combinations thereof.

According to a second aspect of the invention, there is provided a kit comprising at least one primer pair selected from the group consisting of 15 contiguous nucleotides of: TCTCAGTGGGCGTTCTTATG (SEQ ID NO. 1) and TACCCCCTCAACTGCTAATA (SEQ ID NO. 2); TGTCTTCAGCATCTTATGCAG (SEQ ID NO. 3) and CATGATTAATTACTGAAACAGAAAC (SEQ ID NO. 4); GCGGTTTTATTTGCATTAGC (SEQ ID NO. 5) and TCCCGTCAACCTTCACTGTA (SEQ ID NO. 6); GCGGTTTTATTTGCATTAGT (SEQ ID NO. 7) and AGTACTCTTTTCCGGCCACT (SEQ ID NO. 8); ATGAAGTGTATATTGTTAAAGTGGA (SEQ ID NO. 9) and AGCCACATATAAATTATTTCGT (SEQ ID NO. 10); ATGCTTAGTGCTGGTTTAGG (SEQ ID NO. 11) and GCCTTCATCATTTCGCTTTC (SEQ ID NO. 12); GGTAAAATTGAGTTCTCTAAGTAT (SEQ ID NO. 13) and CAGCAAATCCTGAACCTGACG (SEQ ID NO. 14); AGCTGCAAGTGCGGGTCTG (SEQ ID NO. 15) and TACGGGTTATGCCTGCAAGTTCAC (SEQ ID NO. 16); CTACAGGTGAAGGTGGAATGG (SEQ ID NO. 17) and ATTCCTCTCTTTCCTCTGCGG (SEQ ID NO. 18); TACCATCGCAAAAGCAACTCC (SEQ ID NO. 19) and GTCGGCAACGTTAGTGATACC (SEQ ID NO. 20); CCCCCTGGACGAAGACTGAC (SEQ ID NO. 21) and ACCGCTGGCAACAAAGGATA (SEQ ID NO. 22) and combinations thereof.

According to a third aspect of the invention, there is provided a method of detecting the presence or absence of E. coli virulence-related genes in a sample comprising:
adding the sample to an amplification mixture including at least one pair of primers selected from the group consisting of at least 15 contiguous nucleotides of: TCTCAGTGGGCGTTCTTATG (SEQ ID NO. 1) and TACCCCCTCAACTGCTAATA (SEQ ID NO. 2); TGTCTTCAGCATCTTATGCAG (SEQ ID NO. 3) and CATGATTAATTACTGAAACAGAAAC (SEQ ID NO. 4); GCGGTTTTATTTGCATTAGC (SEQ ID NO. 5) and TCCCGTCAACCTTCACTGTA (SEQ ID NO. 6); GCGGTTTTATTTGCATTAGT (SEQ ID NO. 7) and AGTACTCTTTTCCGGCCACT (SEQ ID NO. 8); ATGAAGTGTATATTGTTAAAGTGGA (SEQ ID NO. 9) and AGCCACATATAAATTATTTCGT (SEQ ID NO. 10); ATGCTTAGTGCTGGTTTAGG (SEQ ID NO. 11) and GCCTTCATCATTTCGCTTTC (SEQ ID NO. 12); GGTAAAATTGAGTTCTCTAAGTAT (SEQ ID NO. 13) and CAGCAAATCCTGAACCTGACG (SEQ ID NO. 14); AGCTGCAAGTGCGGGTCTG (SEQ ID NO. 15) and TACGGGTTATGCCTGCAAGTTCAC (SEQ ID NO. 16); CTACAGGTGAAGGTGGAATGG (SEQ ID NO. 17) and ATTCCTCTCTTTCCTCTGCGG (SEQ ID NO. 18); TACCATCGCAAAAGCAACTCC (SEQ ID NO. 19) and GTCGGCAACGTTAGTGATACC (SEQ ID NO. 20); CCCCCTGGACGAAGACTGAC (SEQ ID NO. 21) and ACCGCTGGCAACAAAGGATA (SEQ ID NO. 22) and combinations thereof;
incubating the amplification mixture under conditions which promote DNA amplification; and
identifying the amplification products.

### BRIEF DESCRIPTION OF THE DRAWINGS

FIG. 1 shows the amplification products from the multiplex PCR reaction (1-A is Set A; 1-B is Set B and 1-C is Set C).

TABLE 1 shows the E. coli virulence-associated genes.

TABLE 2 shows the primer sequences and expected sizes of amplification products.

TABLE 3 shows the verocytotoxin results from the multiplex PCR analysis.

TABLE 4 shows the predicted sizes of restriction fragments and enzymes used for restriction fragment length polymorphism analysis of amplified products of multiplex PCR.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Definitions

As used herein, “amplification reaction mixture” or “amplification mixture” refers to an aqueous solution comprising the various reagents used to amplify a target nucleic acid. These include but are by no means limited to enzymes, aqueous buffers, salts, target nucleic acid and nucleoside triphosphates.

As used herein, “isolated” or “substantially pure”, when referring to nucleic acids, refers to those which have been purified away from other cellular components and/or contaminants by standard techniques, for example, column chromatography, CsCl banding, and alkaline/SDS treatment as well as other techniques well known in the art.

As used herein, ‘DNA sequence’ refers to a single-stranded or double-stranded DNA polymer composed of the nucleotide bases, adenosine, thymidine, cytosine and guanosine.

As used herein, “nucleotide polymerase” refers to enzymes that are capable of catalyzing the synthesis of DNA or RNA from nucleoside triphosphate precursors.

As used herein, “primer” refers to an oligonucleotide capable of acting as a point of initiation of DNA synthesis under conditions in which synthesis of a primer extension product complementary to a nucleic acid strand is initiated.

Proper annealing conditions depend, for example, on the length of the primer or probe, the base composition of said primer or probe and the number of mismatches present and their relative position.

Described herein is are a plurality of primer pairs which may be used alone or in combination for example in 3 multiplex PCR assays that can detect in E. coli the presence of the 8 virulence genes: eaeA, EHEC-HlyA, Stx1 (VT1), Stx2 (VT2), Stx2c (VT2c), Stx2d (VT2d), Stx2e (VT2e) and Stx2f (VT2f. In addition, the kit can detect the two critical serotypes (O157 and H7) and identify the species (Escherichia coli) simultaneously using a one step reaction. Following evaluation in our hands, this PCR kit has been used to detect the above 11 components of disease-causing E. coli in a fast, accurate, reliable and specific fashion. These kits can be used on bacterial isolates and has the potential for use directly on foods and environmental samples.

As described above, E. coli O157:H7 is a major cause of both outbreaks and sporadic cases of human diarrheal disease in North America and throughout the world (Griffin and Boyce, 1998, Escherichia coli O157:H7. Emerging infections in Scheld et al., (ASM Press: Washington, D.C.) pp 1347-145; Sparling, 1998, JAVMA 213: 1733; Spika et al, 1998, in E. coli O157:H7 and Other Shiga-Producing E. coli Strains (ASM Press: Washington, D.C.), pp 23-29). Clinical symptoms of the disease may include bloody diarrhea and HC, along with complications associated with HUS, acute and chronic kidney disease, thrombotic thrombocytopenic purpura (TTP), neurologic sequelae and death (Boyce et al., 1995, Current Concepts 333: 364-368; Carter et al., 1987, New Engl J Med 317: 1496-1500; Altekruse et al., 1997, Emerg Infect Dis 3: 285-293; Karmali et al., 1985, J Infect Dis 151: 775-782) Disease associated with E. coli O157:H7 was first reported in the late 1970s; however, the severity of the conditions due to this bacterial pathogen was fully recognized following a spectacular outbreak associated with a fast-food restaurant chain in the US in 1993. Since then, major outbreaks have occurred worldwide and 70-80% of sporadic cases of classic HUS reported in Canada, the United Kingdom, Germany, Belgium, the Netherlands and Japan are caused by infections with this organism (Boyce et al., 1995). Initial cases of infection with VTEC were predominately associated with undercooked ground beef (hamburger) and although this continues to be a major source of infection, new vehicles for transmission of disease include unpasteurized milk, unpasteurized apple juice, salami, alfalfa sprouts, lettuce and untreated water as well as recreational water. These have emerged in recent years to pose major health threats to populations worldwide.

VTEC or Shiga-toxigenic E. coli (STEC), including O157:H7 and other non-O157 serogroups, produce a plethora of toxins that result in human disease. Those major toxins detected to date are shown in Table 1. The VT toxins produce profound cytopathic effects in vero cells and VT1 shows a high degree of homology to the Shiga-toxin (Stx) of Shigella dysenteriae type 1. In outbreaks reported between 1982 and 1983, 23% of patients were hospitalized, 6% developed HUS or TTP and 1.2% died. In 1982, two outbreaks of disease occurred in USA, followed in the same year by a further outbreak in Canada. It was this latter outbreak that led to the recognition of a new pathogen serotype, E. coli O157:H7. In 1985, Canada experienced one of its worst outbreaks to date of E. coli O157:H7. This outbreak occurred in a nursing home and resulted in the deaths of 17 elderly residents.

Foodborne illnesses have a major public health impact worldwide and it has been estimated that in the US alone, 76 million people become ill and more than 325,000 are hospitalized with 5,000 deaths. A significant proportion of these are due to E. coli. In Canada, it has been estimated that there are approximately 30,000 cases of VTEC resulting in 15 to 25 deaths each year. Therefore, differentiation of the causal agents of these diseases is crucial to clinical treatment and patient recovery. Indeed, in the case of E. coli disease, poor diagnosis leading to incorrect treatment in the form of inappropriate antibiotic administration may make the situation worse by killing the infectious agents and releasing kidney-damaging toxins into the blood stream and thus leading to HUS.

E. coli O157:H7 has been isolated from new sources and in increasing numbers as the cause of human infection. Major outbreaks of VTEC O157:H7 have been associated with ground beef, unpasteurized milk, unpasteurized apple juice, salami, alfalfa sprouts, lettuce and untreated water. Indeed, in 2000, a major outbreak of E. coli O157:H7 disease occurred in Ontario and was associated with contaminated drinking water. In all there were 1,346 reported cases of illness identified. Of these, 167 were laboratory confirmed as E. coli O157:H7, 27 developed HUS and 6 people died. Globalization of the food supply has increased the potential for outbreaks of E. coli O157:H7 disease in food products on a worldwide basis. This wide distribution of VTEC in foods and contaminated water, along with the ability of the bacterial toxins to induce severe human disease, makes the development of effective and rapid processes for toxin detection in these organisms absolutely essential for disease control. In addition, the implementation of proper food handling practices and public education on food safety are critical to reducing the disease burden in the population.

Surveillance for pathogens and early identification of outbreaks are also critical for reducing the incidence of foodborne disease. The Canadian National Laboratory for Enteric Pathogens (NLEP) uses surveillance and laboratory-based epidemiologic markers for specific bacteria strains to track human infections and to identify and characterize outbreaks. Currently, E. coli O157:H7 and isolates of non-O157 VTEC are confirmed using biochemical and serological identification techniques; procedures that are complex, slow and outmoded. Typically, isolates are also examined for the production of verotoxins and their associated virulence genes. Further subtyping of VTEC O157 using molecular typing provides key epidemiologic markers for tracing sources of bacteria responsible for human disease, for trace back analysis, and for supporting food or product recalls. Verotoxin genotypes and other VTEC and EHEC virulence factors (e.g. eae and EHEC-HlyA hemolysin genes) are determined using PCR techniques.

Newer molecular models are urgently required for use in clinical and laboratory medicine as well as in the environmental, abbatoir and food industry arenas, to assist in resolving the problem of E. coli disease. This led us to investigate the potential for a rapid, simple and cost effective kit to both identify the serotype of the causal agent (i.e. O157:H7 the major culprit in E. coli disease, otherwise known as “hamburger disease”) as well as to detect the key toxins of E. coli in a one step assay.

As will be known to one of skill in the art, DNA amplification involves allowing two primers to anneal to opposite strands of a template DNA in an amplification mixture and allowing extension of the primers. This process is repeated several times, thereby producing an amplification product. The PCR process is discussed in detail in for example U.S. Pat. No. 4,199,559, U.S. Pat. No. 4,683,195 and U.S. Pat. No. 4,683,202, which are incorporated herein by reference.

To begin the PCR process, the target nucleic acid in the sample is denatured, typically by heating. Once the strands are separated, the next step involves hybridizing the separated strands with the amplification primers. The primers are then extended to form complementary copies of the target strands, and the cycle of denaturation, hybridization and extension is repeated as many times as necessary to obtain the desired amount of amplified nucleic acid.

Template-dependent extension of primers in PCR is catalyzed by a polymerizing agent in the presence of adequate amounts of four deoxyribonucleotide triphosphates in a reaction medium. Suitable polymerizing agents are enzymes known to catalyze template-dependent DNA synthesis. For example, if the template is RNA, a suitable polymerizing agent to convert RNA to cDNA is reverse transcriptase, such as avian myeloblastosis virus RT or Murine Moloney Leukemia Virus RT. If the template is DNA, suitable polymerases include for example E. coli DNA polymerase I, the Klenow fragment of DNA polymerase I, T₄ DNA polymerase, Hot Tub® and Taq polymerase.

A preferred mode for carrying out the PCR reaction is the multiplex mode. The multiplex mode involves the simultaneous amplification of different target regions using more than one set of PCR primers. As will be apparent to one of skill in the art, increasing the number of distinct primers in an amplification mixture can result in production of non-diagnostic bands. As such, the nucleic acid composition, length of the primers selected and the relative position of the primers is critical for functioning of this method.

Referring to Table 2, the primers for use in the multiplex PCR system described herein comprise at least 15 contiguous nucleotides of the following:
TCTCAGTGGGCGTTCTTATG, designated hereafter as SEQ ID NO. 1 or VT1-a. As can be seen, these sequence corresponds to nucleotides 777-796 of Genbank accession No. M17358.
TACCCCCTCAACTGCTAATA, designated hereafter as SEQ ID NO. 2 or VT1-b. As can be seen, these sequence corresponds to nucleotides 1114-1095 of Genbank accession No. M17358.
TGTCTTCAGCATCTTATGCAG, designated hereafter as SEQ ID NO. 3 or VT2F-a. As can be seen, these sequence corresponds to nucleotides 300-320 of Genbank accession No. M29153.
CATGATTAATTACTGAAACAGAAAC, designated hereafter as SEQ ID NO. 4 or VT2F-b. As can be seen, these sequence corresponds to nucleotides 449425 of Genbank accession No. M29153.
GCGGTTTTATTTGCATTAGC, designated hereafter as SEQ ID NO. 5 or VT2-a. As can be seen, these sequence corresponds to nucleotides 1228-1247 of Genbank accession No. X07865.
TCCCGTCAACCTTCACTGTA, designated hereafter as SEQ ID NO. 6 or VT2-b. As can be seen, these sequence corresponds to nucleotides 1342-1323 of Genbank accession No. X07865.
GCGGTTTTATTTGCATTAGT, designated hereafter as SEQ ID NO. 7 or VT2c-a. As can be seen, these sequence corresponds to nucleotides 1186-1205 of Genbank accession No. M59432.
AGTACTCTTTTCCGGCCACT, designated hereafter as SEQ ID NO. 8 or VT2c-b. As can be seen, these sequence corresponds to nucleotides 1309-1290 of Genbank accession No. M59432.
ATGAAGTGTATATTGTTAAAGTGGA, designated hereafter as SEQ ID NO. 9 or VT2e-a. As can be seen, these sequence corresponds to nucleotides 204-228 of Genbank accession No. M36727.
AGCCACATATAAATTATTTCGT, designated hereafter as SEQ ID NO. 10 or VT2e-b. As can be seen, these sequence corresponds to nucleotides 506-485 of Genbank accession No. M36727.
ATGCTTAGTGCTGGTTTAGG, designated hereafter as SEQ ID NO. 11 or EAE-a. As can be seen, these sequence corresponds to nucleotides 132-151 of Genbank accession No. Z11541.
GCCTTCATCATTTCGCTTTC, designated hereafter as SEQ ID NO. 12 or EAE-b. As can be seen, these sequence corresponds to nucleotides 379-360 of Genbank accession No. Z11541.
GGTAAAATTGAGTTCTCTAAGTAT, designated hereafter as SEQ ID NO. 13 or VT2d-a. As can be seen, these sequence corresponds to nucleotides 1221-1244 of Genbank accession No. AF043627.
CAGCAAATCCTGAACCTGACG, designated hereafter as SEQ ID NO. 14 or VT2d-b. As can be seen, these sequence corresponds to nucleotides 1395-1375 of Genbank accession No. AF043627.
AGCTGCAAGTGCGGGTCTG, designated hereafter as SEQ ID NO. 15 or HlyA-a. As can be seen, these sequence corresponds to nucleotides 867-885 of Genbank accession No. X79839.
TACGGGTTATGCCTGCAAGTTCAC, designated hereafter as SEQ ID NO. 16 or HlyA-b. As can be seen, these sequence corresponds to nucleotides 1435-1412 of Genbank accession No. X79839.
CTACAGGTGAAGGTGGAATGG, designated hereafter as SEQ ID NO. 17 or rfbE-a. As can be seen, these sequence corresponds to nucleotides 673-693 of Genbank accession No. S83460.
ATTCCTCTCTTTCCTCTGCGG, designated hereafter as SEQ ID NO. 18 or rfbE-b. As can be seen, these sequence corresponds to nucleotides 999-979 of Genbank accession No. S83460.
TACCATCGCAAAAGCAACTCC, designated hereafter as SEQ ID NO. 19 or flic-a. As can be seen, these sequence corresponds to nucleotides 1068-1088 of Genbank accession No. AF228488.
GTCGGCAACGTTAGTGATACC, designated hereafter as SEQ ID NO. 20 or flic-b. As can be seen, these sequence corresponds to nucleotides 1314-1294 of Genbank accession No. AF228488.
CCCCCTGGACGAAGACTGAC, designated hereafter as SEQ ID NO. 21 or e16S-a. As can be seen, these sequence corresponds to nucleotides 1682-1701 of Genbank accession No. AB035924.
ACCGCTGGCAACAAAGGATA, designated hereafter as SEQ ID NO. 22 or e16S-b. As can be seen, these sequence corresponds to nucleotides 2082-2063 of Genbank accession No. AB035924.

As will be appreciated by one of skill in the art, the primers may comprise at least 16 contiguous nucleotides, that is, 16 or more contiguous nucleotides, at least 17 contiguous nucleotides or at least 18 contiguous nucleotides of any one of the above-described primers. In yet other embodiments, the primers may consist essentially of at least 15 contiguous nucleotides, at least 16 contiguous nucleotides, at least 17 contiguous nucleotides or at least 18 contiguous nucleotides of any one of the above-described primers. As will be appreciated by one of skill in the art, in this context, “consists essentially of” indicates that the primer consists of those nucleotides only but may also include other components which do not materially affect the functioning of the primer (that is, its ability to hybridize to its target sequence). These include for example but are by no means limited to labels, universal bases, tags and the like known in the art.

As described below, the last two primers (SEQ ID Nos 21 and 22) are used as positive controls. As will be apparent to one of skill in the art, other suitable primers which generate an amplification product without producing background or false positive products may also be used as positive controls and are within the scope of the invention.

In use, a sample suspected of E. coli contamination is prepared for PCR analysis. As will be appreciated by one knowledgeable in the art, samples may be selected from any source wherein E. coli contamination is suspected, for example, but by no means limited to, fecal samples, environmental samples, veterinary samples, medical diagnostic samples and food samples. Examples of environmental samples include for example drinking water and recreational water. Examples of food samples include for example ground beef, milk, apple juice, salami, alfalfa sprouts and lettuce as well as any other food product suspected of contamination with E. coli O157:H7. In some embodiments, the sample may be incubated under conditions known in the art which promote amplification of bacteria prior to preparation for PCR analysis.

The sample is then mixed with at least one of the primer pairs described above as well as amplification enzymes, aqueous buffers, salts, target nucleic acid and nucleoside triphosphates as discussed above, thereby forming an amplification mixture. The amplification mixture is then subjected to conditions suitable for nucleic acid amplification. Specifically, as discussed above, nucleic acid in the sample is denatured by heating. Once the strands are separated, the temperature of the sample is lowered and the amplification primers hybridize to their target DNA. The temperature is elevated and the primers are then extended to form complementary copies of the target strands. The cycle of denaturation, hybridization and extension is repeated as many times as necessary to obtain the desired amount of amplified nucleic acid.

The amplification products generated as described above may be detected by any suitable means known in the art, for example, by a characteristic size as detected on a polyacrylamide or agarose gel stained with ethidium bromide. Alternatively, amplified products may be detected by a labeled probe. The label may be for example a radiolabel or a fluorescent or chemiluminescent label. Examples of detection methods known in the art include but are by no means limited to U.S. Pat. No. 6,245,514 and U.S. Pat. No. 6,117,635, both of which are incorporated herein by reference.

As discussed above, the amplification mixture may contain at least one of the above-described primer pairs. In use, an approximately 338 bp fragment indicates the presence of VT1 in the sample when using VT1-a/VT1-b; an approximately 150 bp fragment indicates the presence of VT2f in the sample when using VT2F-a/VT2F-b; an approximately 115 bp fragment indicates the presence of VT2 in the sample when using VT2-a/VT2-b; an approximately 124 bp band indicates the presence of VT2c when using VT2c-a/VT2c-b; an approximately 303 bp band indicates the presence of VT2e when using VT2e-a/VT2e-b; an approximately 248 bp band indicates the presence of eaeA when using EAE-a/EAE-b; an approximately 175 bp band indicates the presence of VT2d when using VT2d-a/VT2d-b; an approximately 569 bp band indicates the presence of EHEH-HlyA when using HlyA-a/HlyA-b; an approximately 327 bp band indicates the presence of rfbE when using rfbE-a/rfbE-b and an approximately 247 bp band indicates the presence of flic when using flic-a/flic-b. It is of note that in some instances, a positive control, for example, primer pair e16S-a/e16S-b, may be included in the amplification mixture to ensure that conditions suitable for DNA amplification were attained.

In one embodiment of the invention, primer pairs VT1-a/VT1-b, VT2F-a/VT2F-b, and VT2-a/VT2-b are mixed with the sample in the amplification mixture. In this embodiment, the amplification products from the amplification mixture may be identified by polyacrylamide and/or agarose gel electrophoresis, wherein an approximately 338 bp fragment indicates the presence of VT1 in the sample; an approximately 150 bp fragment indicates the presence of VT2f in the sample; and an approximately 115 bp fragment indicates the presence of VT2 in the sample. It is of note that in some instances, a positive control, for example, primer pair e16S-a/e16S-b, may be included in the amplification mixture to ensure that conditions suitable for DNA amplification were attained.

In another embodiment of the invention, primer pairs VT2c-a/VT2c-b, VT2e-a/VT2e-b, and EAE-a/EAE-b are mixed with the sample in an amplification mixture. In this embodiment, the presence of an approximately 124 bp band on a polyacrylamide and/or agarose gel indicates the presence of VT2c; the presence of an approximately 303 bp band indicates the presence of VT2e; and the presence of an approximately 248 bp band indicates the presence of eaeA. It is of note that in some instances, a positive control, for example, primer pair e16S-a/e16S-b, may be included in the amplification mixture to ensure that conditions suitable for DNA amplification were attained.

In another embodiment of the invention, primer pairs VT2d-a/VT2d-b, HlyA-a/HlyA-b, rfbE-a/rfbE-b and flic-a/flic-b are mixed together with the sample in an amplification mixture. In this embodiment, the presence of an approximately 175 bp band on a polyacrylamide and/or agarose gel indicates the presence of VT2d; the presence of an approximately 569 bp band indicates the presence of EHEH-HlyA; the presence of an approximately 327 bp band indicates the presence of rfbE; and the presence of an approximately 247 bp band indicates the presence of flic. It is of note that in some instances, a positive control, for example, primer pair e16S-a/e16S-b, may be included in the amplification mixture to ensure that conditions suitable for DNA amplification were attained.

As will be appreciated by one knowledgeable in the art, the exact sizes of the amplification products described above may vary somewhat depending on the specific sequences of the primer pairs utilized.

For commercial convenience, some or all of the above-described primers may be packaged in the form of a kit. That is, the kit will include at least one pair of primers selected from the group consisting of VT1-a/VT1-b, VT2F-a/VT2F-b, VT2-a/VT2-b, VT2c-a/VT2c-b, VT2e-a/VT2e-b, EAE-a/EAE-b VT2d-a/VT2d-b, HlyA-a/HlyA-b, rfbE-a/rfbE-b, flic-a/flic-b and combinations thereof. Reagents for performing a nucleic acid amplification reaction may also be included with the amplification primers, for example, buffers, additional primers, positive and negative controls, nucleoside triphosphates, enzymes, and instructions. For example, the kit may include template DNA that will hybridize to each individual primer pair as a positive control. In one embodiment, the kit may comprise primer pairs VT1-a/VT1-b, VT2F-a/VT2F-b, and VT2-a/VT2-b as well as optionally a positive control as discussed above. In another embodiment, the kit may comprise primer pairs VT2c-a/VT2c-b, VT2e-a/VT2e-b, and EAE-a/EAE-b and optionally a suitable positive control. In another embodiment of the invention, the kit may comprise primer pairs VT2d-a/VT2d-b, HlyA-a/HlyA-b, rfbE-a/rfbE-b and flic-a/flic-b as well as optionally a positive control.

As discussed below, in one embodiment, the PCR products are visualized on a gel following electrophoretic separation. As will be appreciated by one knowledgeable in the art, in some embodiments, detection of the bands may be automated wherein the samples are loaded onto a suitable separating system and bands are detected automatically. Examples of such techniques may be found in for example U.S. Pat. No. 5,840,877, U.S. Pat. No. 4,930,893, U.S. Pat. No. 6,005,663, U.S. Pat. No. 5,710,628, U.S. Pat. No. 5,543,018 and U.S. Pat. No. 5,190,632, which are incorporated herein by reference.

It is of note that in some embodiments, separation systems arranged to resolve relatively small differences between nucleic acid molecules may be used. As will be apparent to one of skill in the art, this would allow resolution of amplification products having similar sizes.

It is of note that any amplification protocol which utilizes cyclic, specific hybridization of primers to the target sequence, extension of the primers using the target sequence as a template and separation or displacement of the extension products from the target sequence may employ the amplification primers described herein.

The invention will now be described by way of examples. However, the invention is not limited to the examples.

### EXAMPLE I

### Bacterial Strains and Culture Media

A total of 129 E. coli isolates from the culture collection of the National Laboratory for Enteric Pathogens (NLEP) were used in this study and included: 79 E. coli O157:H7, 5 O157:NM (non-motile), 7 O157:non-H7 (one each of O157:H10, H19, H21, H43, H45 and 2 H16), 12 non-O157:H7 (2 O27:H7, 3 O18:H7, 5 O55:H7, 1 each of 156:H7 and O83:H7), 6 non-O157:NM (1 each of O1:NM, O7:NM, O91:NM, OR(rough):NM and 2 O111:NM), 14 non O157:non-H7 (1 O6:H1, 2 O103:H2, 1 O146:H21, 1 O26:H11, 1 O70:H11, 1 O91:H21, 1 O139:K82, 1 O128:B12 and 1 O15:H27, 2 O128:H?, 1 O113:21, 1 OR:H21), 3 O UT(untypable):H7, 1 O UT:H8 and 2 O UT: H UT. Of these, 101 strains were VTEC and 28 were VT negative. The control strains had been previously defined in terms of virulence factors and toxigenicity with respect to VT1, VT2, VT2c, VT2+VT2c, VT2d, VT2e, VT2f, eaeA and EHEC-hlyA (Table 1).

### EXAMPLE II

### DNA Isolation

Total DNA was isolated from 0.5 ml of brain heart infusion broth culture grown overnight for all the bacterial strains used in this study. The procedure used for DNA isolation was described in Tyler et al., 1991, J Clin Microbiol 29: 1339-1343, which is incorporated herein by reference. DNA samples were dissolved in Tris-EDTA buffer (10 mM Tris, i mM EDTA [pH 8.0]), and the concentration was determined in μg/ml at an optical density reading of A260. Template DNA concentration used was 2 μg/ml.

### EXAMPLE III

### Primers

Oligonucleotides ranging from 19 to 25 mers were selected as described above. Synthesis of oligonucleotides was carried out at the DNA Core Facility at the National Microbiology Laboratory, Winnipeg, Canada. As discussed above, for multiplex PCR, 3 primer sets were prepared: Set A which was designed to amplify VT1 (VT1-a/VT1-b), VT2 (VT2-a/VT2-b), VT2f (VT2F-a/VT2F-b) and 16S rRNA; Set B which was designed to amplify VT2c (VT2c-a/VT2c-b), VT2e (VT2e-a/VT2e-b), eaeA (EAE-a/EAE-b) and 16S rRNA; and Set C which was designed to amplify VT2d (VT2d-a/VT2d-b), EHEC-hlyA (HlyA-a/HlyA-b), rfbE (rfbE-a/rfbE-b), flic (flic-a/flic-b) and 16S rRNA. The primer sequences are described above and in Table 2.

### EXAMPLE IV

### Multiplex PCR Conditions

Three sets (A, B and C) of primer mixtures were prepared according to the AmpliTaq™ Gold kit (Applied Biosystems, Forster City, Calif.), with slight modifications to the given instructions. In general, all of the multiplex primer sets contained 200 μM deoxynucleoside triphosphates; 2.5 μl of 10× reaction buffer (100 mM Tris-HCl [pH 8.3], 500 mM KCl); 1.5 mM MgCl₂ and 0.1 μM of 16S rRNA primers. Set A contained 0.5 μM (each) of VT1-a/VT1-b, VT2F-a/VT2F-b, and VT2-a/VT2-b; 2.5 U of Taq DNA polymerase (AmpliTaq Gold; Applied Biosystems, Forster City Calif.), and 5 ng of template DNA. This mix was brought to 25 μl with sterile water. Multiplex primer Set B included the same constituents as Set A except for the primers, which were 1.5 μM of VT2c-a/VT2c-b, 0.4 μM of VT2e-a/VT2e-b, and 0.75 μM of EAE-a/EAE-b. The primers for multiplex primer Set C were 1.5 μM VT2d-a/VT2d-b, 1.0 μM HlyA-a/HlyA-b, 1.0 μM rfbE-a/rfbE-b and 0.4 μM flic-a/flic-b. It is of note that other combinations may also be used, as discussed herein. DNA amplification was carried out in a Perkin-Elmer thermocycler™ 2400 using an initial denaturation step at 95° C. for 8 min, followed by 30 cycles of amplification (denaturation at 95° C. for 30 seconds, annealing at 58° C. for 30 seconds and extension at 72° C. for 30 seconds), ending with a final extension at 72° C. for 7 minutes. It is of note that other suitable primer concentrations, times and temperatures may also be used.

### EXAMPLE V

### Results

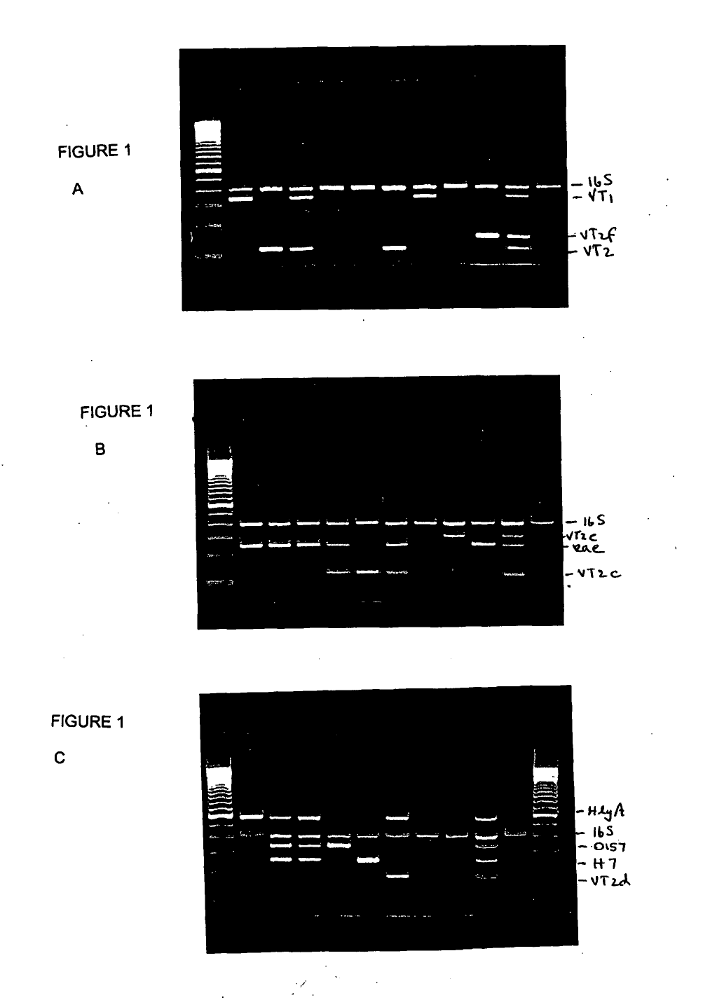

The primers were designed to target the coding regions of the genes and care was taken to avoid areas of homology within the structural genes for the VT2 family. FIG. 1 shows the presence of the amplified product profiles after agarose gel electrophoresis, when DNA extracted from a reference E. coli strain (positive control) was used as the template in the PCR reaction using the multiplex primer sets. Reliable amplification of 4 bands in Set A (VT1, VT2, VT2f and 16S rRNA) were obtained when a mixture of DNA from the same strains were tested (FIG. 1-A). Similarly, 4 bands were obtained when a mixture of DNA from the corresponding strains (VT2c, eaeA, VT2e and 16S rRNA) in Set B were tested (FIG. 1-B). Similarly, for Set C, which consisted of VT2d, EHEC-hlyA, rfbE, flic and 16S rRNA, a total of 5 bands were obtained for the positive control DNA (FIG. 1-C). As can be seen, the various control strains corresponded to the predicted sizes as discussed above and as shown in Table 2. As a negative control, all sets were tested with E. coli strain ATCC 25922 in which only the 16S rRNA band was observed (lane 12 in FIGS. 1-A and 1-B and lane 11 in FIG. 1-C). Genomic DNA from Aeromonas hydrophilia and Campylobacter jejuni were also tested using these three primer sets and none showed specific PCR amplification.

To substantiate the multiplex PCR technique, 129 strains of E. coli that were tested by multiplex PCR were also screened for the presence of individual toxin genes by using the methods described previously (Hu et al., 1999, J Appl Microbiol 87: 867-876; Johnson et al., 1990, J Clin Microbiol 28: 2351-2353; Johnson et al., 1991, FEMS Microbiol Lett 68: 227-230; Meng et al., 1997, Lett Appl Microbiol 24: 172-176; Paton and Paton, 1997, J Clin Micro 36: 598-602). VT2 subtype VT2c and VT2d were confirmed by PCR-RFLP (Pierard et al., 1998, J Clin Microbiol 36: 3317-3322; Tyler et al., 1991, J Clin Microbiol 29: 1339-1343), E. coli O157:H7 and other serotype strains were identified at the NLEP. While agreement of toxigenic profile and O157:H7 were observed (Table 3), 3 of the 11 phenotypically NM strains showed positive results for H7 by PCR—one of these was from the reference strain E32511. An internal control of E. coli 16S rRNA was present in all of the samples, confirming the presence and the quality of E. coli DNA as well as validating the PCR conditions.

The sizes of the amplicons or amplification products obtained by the multiplex primer sets were identical to those predicted from the design of the primers (Table 2). The amplicons from the control strains were subjected to further confirmation and characterization by digestion with restriction endonucleases with cleavage sites within the amplicon. The restriction enzymes used and the predicted product sizes are given in Table 4. Enzyme fragments with the anticipated sizes were obtained in each case.

Among the 129 strains tested, 101 (78.3%) were positive for VTs, 96 (74.4%) were positive for the eaeA, while 7 were detected among VT negative strains. All of O157:H7 strains were eaeA and EHEC-hlyA positive. The ability of the C set of primers to identify O157:H7 from other E. coli strains was determined by analyzing 79 O157:H7, 5 O157:NM and 45 non-O157 E. coli isolates. Two of the 5 O157:NM and 1 of the 6 non-O157:NM strains were flic gene positive, indicating that these isolates were genetically H7 with undetectable flagella antigen in serotyping (Table 3) (Fields et al., 1997, J Clin Microbiol 35: 1066-1070). All of the 129 samples tested contained the E. coli 16S rRNA gene.

### EXAMPLE VI

### Discussion

VTEC have been associated with disease outbreaks of HC and HUS in humans. Two main categories of E. coli VT toxins are VT1 and VT2. VT1 is a homogeneous family of toxins identical to the Shiga toxins of Shigella dysenteriae. VT2 is a more heterogeneous family of toxins and serologically distinct from VT1. Within the VT2 toxin family, VT2c was formally subdivided into VT2-Va and VT2-Vb (Ito et al., 1990, Microb Pathog 8: 47-60; Tyler et al., 1991). These are only partially neutralized by antiserum to VT2 (Head et al., 1988, Lancet ii:751; Hii et al., 1991, J Clin Microbiol 29: 2704-2709); VT2d (Paton et al., 1992, Microb Pathog 13: 225-236; Paton et al., 1993, Gene 129: 87-92; Pierard et al., 1998). Vt2e is cytotoxic only in Vero cells and has been associated with porcine edema disease (Gyles et al., 1988, Microb Pathog 5: 419-426; Marques et al., 1987, FEMS Microbiol Lett 44: 33-38). VT2f (also called VTeV) shows low level cytotoxicity in Vero cells and is readily neutralized by antisera against VT2 and VT2e (Gannon et al., 1990, J Gen Microbiol 136: 1125-1135; Schmidt et al, 2000, Appl Envir Microbiol 66: 1205-1208).

Within the VTEC family, certain strains appear to have greater virulence for humans. Epidemiologically, VT2 seems to be more important than VT1 in the development of HUS in humans, in that strains producing VT2 class toxins resulted in HUS more frequently than did those expressing VT1 (Griffin and Tauxe, 1991, Epidemiol Rev 13: 6098). Some data suggest that toxin type could be important in determining the probability of developing HUS. The VT family of toxins, particularly those related to VT2, are a diverse group of toxins which may differ in terms of their in vitro or in vivo properties. Experiments with clones carrying chimeric O48/OX3b VT2 operons indicated that the increased virulence was a function of the A subunit of VT2/OX3b. The latter differs in its A subunit structure from that of VT2/O48 by only two amino acids (Met-4→Thr and Gly-102→Asp, respectively). These findings raise the possibility that naturally occurring VT2 sequence variations may impact directly on the capacity of a given VT-producing E. coli strain to cause disease (Paton et al., 1995, Infect Immun 63: 2450-2458).

The use of multiplex PCR or PCR-RFLP to characterize VT2 and its subtypes has been well documented (Feng and Monday, 2000, Mol Cell Probes 14: 333-337; Fratamico et al., 2000, J Food Prot 63: 1032-1037; Gannon et al., 1997, Adv Exp Med Biol 412: 81-82; Gannon et al., 1997, J Clin Microbiol 35: 656-662; Hu et al., 1999, J Appl Microbiol 87: 867-876; Meng et al., 1997, Lett Appl Microbiol 24: 172-176; Pass et al., 2000, J Clin Microbiol 38: 2001-2004; Pierard et al., 1998; Tyler et al., 1991; Wang et al., 2000, J Clin Microbiol 38: 1786-1790). Lin et al. (Lin et al., 1993, Microbiol Immunol 37: 543-548) introduced common primers for PCR-RFLP in order to detect the genes for various VTs. However, all these PCR related methods require restriction digestion.

We have described a multiplex PCR-based diagnostic protocol to detect the genes for VTs including VT1, VT2, VT2c, VT2d, VT2e, VT2f, eaeA, EHEC-hlyA, O157 (rfbE) and H7 (flic) without the need for enzyme digestion. Compared to the individual primers and PCR-RFLP results, the multiplex PCR primer sets were shown to be highly specific, reliable, and, most importantly, effective in detecting all 11 genes, including the internal control gene. All primers were gene specific, as demonstrated by restriction fragment lengths obtained after specific restriction endonuclease digestion of the amplicons.

In this study, the toxin genotypes and O157:H7 serotype of E. coli strains are demonstrated (Table 3). Of 81 VTEC-O157:H7 clinical isolates (including the 2 that were serotypically O157:NM but were PCR positive for flic), 100% showed eaeA and EHEC-hlyA positive. These findings are in agreement with previous reports (Boerlin et al., 1999, J Clin Microbiol 37: 497-503; Schmidt et al., 1995, Infect Immun 63: 1055-1061). Interestingly, among 20 of the non-0157 VTEC isolates, 8 (40%) showed eaeA and 11 out of 20 (55%) were EHEC-hlyA positive (Table 3). Three of 10 VT2c positive only strains were eaeA and EHEC-hlyA negative while reference VT2c strain (O91:H21, an isolate from a case of HUS) showed hlyA positive and eaeA negative suggesting that the eaeA may not be an essential major virulence factor associated with HUS VT2d stains' genotypes showed that all 4 were eaeA negative. Of the 3 VT1 and VT2d, 2 were hlyA positive indicating that VTEC strains without hlyA may possess reduced pathogenicity or may be non-pathogenic in humans (Stephan and Hoelzle, 2000, Lett Appl Microbiol 31: 139-142). Furthermore, among the 28 non-VTEC isolates (VT negative), 7 were eaeA positive and none possessed the hlyA gene. This implies that EHEC-hlyA may be a more critical virulence factor for disease than eaeA (Table 3).

In total, 11 phenotypical non-motile E. coli isolates were analyzed. Of these, 3 were flic positive. Two of these were O157:NM (including reference strain E32511) and 1 was a O1:NM strain. All 3 were confirmed for H7 positive using primer FLICₕ₇-F and FLICₕ₇-R (Hu et al., 1999). E. coli flic sequence comparison (Genbank Accession No. AF228487-O157:H7, AF228495-O19ab:H7, AF228496-O53:H7, AF228489-O55:H7 and U47614-O157:NM) also confirmed that flic is highly conserved in different serogroups. Therefore, it would appear that some E. coli strains that are serologically NM are genetically H7 (Fields et al., 1997, J Clin Microbiol 35: 1066-1070).

As can be seen, the multiplex primer sets described in this study are specific and give consistent results. The use of this method will allow simultaneous assaying for the major virulence factors in E. coli 0157 and non 0157 strains while avoiding the need for endonuclease digestion.

While the preferred embodiments of the invention have been described above, it will be recognized and understood that various modifications may be made therein, and the appended claims are intended to cover all such modifications which may fall within the spirit and scope of the invention.

TABLE 1
Reference strains' information used in this study

| | | | EaeA/ | |
|---|---|---|---|---|
| Strains | Serotype | VTs genes(SLT; Stx) | hly-A | Reference |
| H19 | O26:H11 | VT1(SLT-I: Stx1) | | 8 |
| 933W | O157:H7 | VT2(SLT-II; Stx2) | | 16 |
| B2F1 | O91:H21 | VT2c(SLT-IIvha, | | 15 |
| | | SLT-IIvhb; Stx2c) | | |
| 86-704 | O15:H27 | VT2c(SLT-IIvhb; | | This study |
| | | Stx2c) | | |
| E32511 | O157:NM | VT2, VT2c(SLT-II, | | 32 |
| | | SLT-IIc) | | |
| 91-126 | O128:H? | VT1 + VT2d(Stx1 + | | This study, |
| | | Stx2d) | | 11 |
| 412 | O139:K82 | VT2e(SLT-IIe; | | 10 |
| | | Stx2e) | | |
| H.I.8 | O128:B12 | VT2f(SLT-IIva; | | 5 |
| | | Stx2f) | | |
| 90-2380 | O157:H7 | VT2 | eaeA, | This study |
| | | | hlyA | |
| 25922 | O6:H1 | — | | ATCC |

TABLE 2

Primers used in this study

| | Primer | | Target | Location | Size of PCR | Genbank |
|---|---|---|---|---|---|---|
| Set | pairs | Sequence (5′ to 3′) | Gene | within gene | amplicon (bp) | accession No. |
| A | VT1-a | TCTCAGTGGGCGTTCTTATG | VT1 | 777-796 | 338 | M17358 |
| | VT1-b | TACCCCCTCAACTGCTAATA | | 1114-1095 | | |
| A | VT2F-a | TGTCTTCAGCATCTTATGCAG | VT2f | 300-320 | 150 | M29153 |
| | VT2F-b | CATGATTAATTACTGAAACAGAAAC | | 449-425 | | |
| A | VT2-a | GCGGTTTTATTTGCATTAGC | VT2 | 1228-1247 | 115 | X07865 |
| | VT2-b | TCCCGTCAACCTTCACTGTA | | 1342-1323 | | |
| B | VT2c-a | GCGGTTTTATTTGCATTAGT | VT2c | 1186-1205 | 124 | M59432 |
| | VT2c-b | AGTACTCTTTTCCGGCCACT | | 1309-1290 | | |
| B | VT2e-a | ATGAAGTGTATATTGTTAAAGTGGA | VT2e | 204-228 | 303 | M36727 |
| | VT2e-b | AGCCACATATAAATTATTTCGT | | 506-485 | | |
| B | EAE-a | ATGCTTAGTGCTGGTTTAGG | EAE | 132-151 | 248 | Z11541 |
| | EAE-b | GCCTTCATCATTTCGCTTTC | | 379-360 | | |
| C | VT2d-a | GGTAAAATTGAGTTCTCTAAGTAT | VT2d | 1221-1244 | 175 | AF043627 |
| | VT2d-b | CAGCAAATCCTGAACCTGACG | | 1395-1375 | | |
| C | HlyA-a | AGCTGCAAGTGCGGGTCTG | EHEH-HlyA | 867-885 | 569 | X79839 |
| | HlyA-b | TACGGGTTATGCCTGCAAGTTCAC | | 1435-1412 | | |
| C | rfbE-a | CTACAGGTGAAGGTGGAATGG | rfbE | 673-693 | 327 | S83460 |
| | rfbE-b | ATTCCTCTCTTTCCTCTGCGG | | 999-979 | | |
| C | Flic-a | TACCATCGCAAAAGCAACTCC | flic | 1068-1088 | 247 | AF228488 |
| | flic-b | GTCGGCAACGTTAGTGATACC | | 1314-1294 | | |
| * | e16S-a | CCCCCTGGACGAAGACTGAC | E.coli 16S rRNA | 1682-1701 | 401 | AB035924 |
| | e16S-b | ACCGCTGGCAACAAAGGATA | | 2082-2063 | | |

* Used in all stes as the internal control.

TABLE 3
Verocytotoxin results by 3 sets multiplex PCR analysis of various E. coli strains

| VT Toxins confirmed by | | A set | | | B set | | | C set | | | |
|---|---|---|---|---|---|---|---|---|---|---|---|
| PCR/PCR-RFLP (n) | Serotypes (n) | VT1 | VT2 | VT2f | VT2c | VT2e | eaeA | VT2d | hlyA | O157 | H7 |
| VT1(13) | Non-O157:Non-H7(5) | 5 | — | — | — | — | 4 | — | 5 | — | — |
| | O11I:NM (1) | 1 | — | — | — | — | 1 | — | 1 | — | — |
| | O91:NM (1) | 1 | — | — | — | — | — | — | 1 | — | — |
| | O157:H7 (5) | 5 | — | — | — | — | 5 | — | 5 | 5 | 5 |
| | O157:NM (1) | 1 | — | — | — | — | 1 | — | 1 | 1 | 1 |
| VT1 + VT2 (27) | O UT:H UT (1) | 1 | 1 | — | — | — | — | — | — | — | — |
| | O157:H7 (26) | 26 | 26 | — | — | — | 26 | — | 26 | 26 | 26 |
| VT1 + VT2c(2) | O157:H7 (1) | 1 | — | — | 1 | — | 1 | — | 1 | 1 | 1 |
| | O111:NM (1) | 1 | — | — | 1 | — | 1 | — | — | — | — |
| VT1 + VT2d (3) | O128:H? (2) | 2 | — | — | — | — | — | 2 | 2 | — | — |
| | O rough:NM (1) | 1 | — | — | — | — | — | 1 | — | — | — |
| VT2 (16) | O157:H7 (16) | — | 16 | — | — | — | 16 | — | 16 | 16 | 16 |
| VT2 + VT2c (27) | O157:NM (1) | — | 1 | — | 1 | — | 1 | — | 1 | 1 | 1 |
| | O157:H7 (26) | — | 26 | — | 26 | — | 26 | — | 26 | 26 | 26 |
| VT2c (10) | O91:H21 (1) | — | — | — | 1 | — | — | — | 1 | — | — |
| | Non-O157:Non-H7 (3) | — | — | — | 3 | — | — | — | 1 | — | — |
| | O157:H7 (5) | — | — | — | 5 | — | 5 | — | 5 | 5 | 5 |
| | O55:H7 (1) | — | — | — | 1 | — | 1 | — | — | — | 1 |
| VT2d (1) | O UT:H8 (1) | — | — | — | — | — | — | 1 | — | — | — |
| VT2e (1) | O139:K82 (1) | — | — | — | — | 1 | — | — | — | — | — |
| VT2f (1) | O128:B12 (1) | — | — | 1 | — | — | 1 | — | — | — | — |
| VT s Negative (28) | O157:Non H7 (7) | — | — | — | — | — | 6 | — | — | 7 | — |
| | O157:NM (3) | — | — | — | — | — | — | — | — | 3 | — |
| | Non-O157:H7 (11) | — | — | — | — | — | 1 | — | — | — | 11 |
| | O UT:H7 (3) | — | — | — | — | — | — | — | — | — | 3 |
| | Non-O157:NM (2) | — | — | — | — | — | — | — | — | — | 1 |
| | Non-O157:Non-H7 (1) | — | — | — | — | — | — | — | — | — | — |
| | O UT:H UT (1) | — | — | — | — | — | — | — | — | — | — |
| Total 101/129 | | 45 | 70 | 1 | 39 | 1 | 89/101 | 4 | 92/101 | 91 | 106 |

TABLE 4
Predicted sizes of restriction fragments and enzymes
used for restriction fragment length polymorphism
analysis of amplified products of multiplex PCR

| | PCR | | | Expected size |
|---|---|---|---|---|
| VTs/ | amplicon | Multiplex | | of restriction |
| genes | sizes(bp) | Primer set | Enzymes | fragments(bp) |
| VT1 | 338 | A | BglI | 136, 202 |
| VT2 | 115 | A | BsrDI | 37, 78 |
| VT2f | 150 | A | AluI | 54, 96 |
| VT2e | 303 | B | TaqI | 51, 87, 165 |
| VT2c | 124 | B | HhaI | 48, 76 |
| eaeA | 248 | B | AluI | 109, 139 |
| VT2d | 175 | C | RsaI | 66, 109 |
| EHEC-hlyA | 569 | C | ApaI | 299, 270 |
| rfbE | 327 | C | AluI | 80, 93, 154 |
| flic | 247 | C | AluI | 40, 207 |
| E. coli | 401 | A/B/C | RsaI | 156, 245 |
| 16S rRNA | | | | |

1. A primer selected from the group consisting of at least 15 contiguous nucleotides of: TCTCAGTGGGCGTTCTTATG (SEQ ID NO. 1); TACCCCCTCAACTGCTAATA (SEQ ID NO. 2); TGTCTTCAGCATCTTATGCAG (SEQ ID NO. 3); CATGATTAATTACTGAAACAGAAAC (SEQ ID NO. 4); GCGGTTTTATTTGCATTAGC (SEQ ID NO. 5); TCCCGTCAACCTTCACTGTA (SEQ ID NO. 6); GCGGTTTTATTTGCATTAGT (SEQ ID NO. 7); GTACTCTTTTCCGGCCACT (SEQ ID NO. 8); ATGAAGTGTATATTGTTAAAGTGGA (SEQ ID NO. 9); AGCCACATATAAATTATTTCGT (SEQ ID NO. 10); ATGCTTAGTGCTGGTTTAGG (SEQ ID NO. 11); GCCTTCATCATTTCGCTTTC (SEQ ID NO. 12); GGTAAAATTGAGTTCTCTAAGTAT (SEQ ID NO. 13); CAGCAAATCCTGAACCTGACG (SEQ ID NO. 14); AGCTGCAAGTGCGGGTCTG (SEQ ID NO. 15); TACGGGTTATGCCTGCAAGTTCAC (SEQ ID NO. 16); CTACAGGTGAAGGTGGAATGG (SEQ ID NO. 17); ATTCCTCTCTTTCCTCTGCGG (SEQ ID NO. 18); TACCATCGCAAAAGCAACTCC (SEQ ID NO. 19); GTCGGCAACGTTAGTGATACC (SEQ ID NO. 20); CCCCCTGGACGAAGACTGAC (SEQ ID NO. 21) and ACCGCTGGCAACAAAGGATA (SEQ ID NO. 22) and combinations thereof.

2. A kit comprising at least one primer selected from the group consisting of at least 15 contiguous nucleotides of: TCTCAGTGGGCGTTCTTATG (SEQ ID NO. 1); TACCCCCTCAACTGCTAATA (SEQ ID NO. 2); TGTCTTCAGCATCTTATGCAG (SEQ ID NO. 3); CATGATTAATTACTGAAACAGAAAC (SEQ ID NO. 4); GCGGTTTTATTTGCATTAGC (SEQ ID NO. 5); TCCCGTCAACCTTCACTGTA (SEQ ID NO. 6); GCGGTTTTATTTGCATTAGT (SEQ ID NO. 7); AGTACTCTTTTCCGGCCACT (SEQ ID NO. 8); ATGAAGTGTATATTGTTAAAGTGGA (SEQ ID NO. 9); AGCCACATATAAATTATTTCGT (SEQ ID NO. 10); ATGCTTAGTGCTGGTTTAGG (SEQ ID NO. 11); GCCTTCATCATTTCGCTTTC (SEQ ID NO. 12); GGTAAAATTGAGTTCTCTAAGTAT (SEQ ID NO. 13); CAGCAAATCCTGAACCTGACG (SEQ ID NO. 14); AGCTGCAAGTGCGGGTCTG (SEQ ID NO. 15); TACGGGTTATGCCTGCAAGTTCAC (SEQ ID NO. 16); CTACAGGTGAAGGTGGAATGG (SEQ ID NO. 17); ATTCCTCTCTTTCCTCTGCGG (SEQ ID NO. 18); TACCATCGCAAAAGCAACTCC (SEQ ID NO. 19); GTCGGCAACGTTAGTGATACC (SEQ ID NO. 20); CCCCCTGGACGAAGACTGAC (SEQ ID NO. 21) and ACCGCTGGCAACAAAGGATA (SEQ ID NO. 22) and combinations thereof.

3. A method of detecting the presence or absence of E. coli virulence-related genes in a sample comprising:
adding the sample to an amplification mixture including at least one pair of primers selected from the group consisting of at least 15 contiguous nucleotides of: TCTCAGTGGGCGTTCTTATG (SEQ ID NO. 1) and TACCCCCTCAACTGCTAATA (SEQ ID NO. 2); TGTCTTCAGCATCTTATGCAG (SEQ ID NO. 3) and CATGATTAATTACTGAAACAGAAAC (SEQ ID NO. 4); GCGGTTTTATTTGCATTAGC (SEQ ID NO. 5) and TCCCGTCAACCTTCACTGTA (SEQ ID NO. 6); GCGGTTTTATTTGCATTAGT (SEQ ID NO. 7) and AGTACTCTTTTCCGGCCACT (SEQ ID NO. 8); ATGAAGTGTATATTGTTAAAGTGGA (SEQ ID NO. 9) and AGCCACATATAAATTATTTCGT (SEQ ID NO. 10); ATGCTTAGTGCTGGTTTAGG (SEQ ID NO. 11) and GCCTTCATCATTTCGCTTTC (SEQ ID NO. 12); GGTAAAATTGAGTTCTCTAAGTAT (SEQ ID NO. 13) and CAGCAAATCCTGAACCTGACG (SEQ ID NO. 14); AGCTGCAAGTGCGGGTCTG (SEQ ID NO. 15) and TACGGGTTATGCCTGCAAGTTCAC (SEQ ID NO. 16); CTACAGGTGAAGGTGGAATGG (SEQ ID NO. 17) and ATTCCTCTCTTTCCTCTGCGG (SEQ ID NO. 18); TACCATCGCAAAAGCAACTCC (SEQ ID NO. 19) and GTCGGCAACGTTAGTGATACC (SEQ ID NO. 20); CCCCCTGGACGAAGACTGAC (SEQ ID NO. 21) and ACCGCTGGCAACAAAGGATA (SEQ ID NO. 22) and combinations thereof;
incubating the amplification mixture under conditions which promote DNA amplification; and
identifying the amplification products.
